(19) United States
(12) Patent Application Publication
(10) Pub. No.: US 2021/0088489 A1
(43) Pub. Date: Mar. 25, 2021

(54) GAS SENSOR AND GAS DETECTION DEVICE

(71) Applicant: SHARP KABUSHIKI KAISHA, Sakai City, Osaka (JP)

(72) Inventors: NOBORU IWATA, Sakai City (JP); TATSUHITO ARIMURA, Sakai City (JP); MOTOTAKA TANEYA, Sakai City (JP)

(73) Assignee: SHARP KABUSHIKI KAISHA, Sakai City, Osaka (JP)

(21) Appl. No.: 16/096,686

(22) PCT Filed: Dec. 20, 2016
(86) PCT No.: PCT/JP2016/087961

§ 371 Date: Oct. 25, 2018

(30) Foreign Application Priority Data
Apr. 27, 2016 (JP) 2016-089444

(51) Int. Cl. G01N 33/00; G01N 27/12

(52) U.S. Cl. CPC G01N 33/0031; G01N 27/122

(57) ABSTRACT

Gas detection performance is enhanced with a configuration that does not need an air current generating device. In a gas sensor, three or more gas sensor elements are arranged such that two or more gas sensor elements have substantially the same gas response characteristic, and a first phantom line connecting two of the elements intersects an element except for the elements forming the first phantom line or intersects at least one second phantom line formed by elements except for the elements forming the first phantom line.

### TECHNICAL FIELD

The present disclosure relates to a gas sensor that detects gas included in an air current and a gas detection device including the gas sensor installed therein.

### BACKGROUND ART

Many gas sensors for detecting gas included in an air current are being developed. The gas sensors include a plurality of gas sensor elements having different gas response characteristics, and can thus identify a type and a concentration of gas based on a detection result of each of the gas sensor elements. FIG. 18 is a diagram illustrating an arrangement example of gas sensor elements in a gas sensor in the related art. As illustrated, gas sensor elements W to Z having different gas response characteristics are arranged in one line with respect to a direction in which an air current flows in the related art (for example, PTL 1).

### CITATION LIST

### Patent Literature

PTL 1: JP 03-289555 A (published on Dec. 19, 1991)

PTL 2: JP 2001-201436 A (published on Jul. 27, 2001)

PTL 3: JP 2000-171424 A (published on Jun. 23, 2000)

PTL 4: JP 2005-030909 A (published on Feb. 3, 2005)

### SUMMARY OF INVENTION

### Technical Problem

In the gas sensor according to the related art, when timing of gas detection is off in each of the gas sensor elements due to a difference in time at which an air current reaches each of the gas sensor elements (timing of arrival), false detection of a type of gas and a detection error of a concentration occur. An air current is often weak and. unstable particularly in the absence of an air current source near the gas sensor. Thus, a. deviation in the above-mentioned gas detection timing becomes noticeable, thereby making the above-described problem noticeable as well.

However, in a case where an air current source is installed near the gas sensor to solve this problem, such a disadvantage arises that power consumption, a size, and a production cost of the entire device and system for gas detection increase. Further, when an air current source is installed near the gas sensor to artificially generate an air current, a speed and a direction of the air current that needs to be identified in the first place are changed. Thus, a new problem arises that a speed and a direction of an air current included in gas cannot be identified.

The present disclosure has been made in view of the problems, and an object thereof is to enhance gas detection performance with a configuration that does not need an air current source.

### Solution to Problem

To solve the above-described problem, a gas sensor according to the present disclosure includes: three or more detectors configured to detect gas included in an air current. Two or more detectors of the detectors have substantially the same gas response characteristic, The detectors are arranged such that a first phantom line connecting two detectors of the detectors having substantially the same gas response characteristic intersects another detector except for the detectors forming the first phantom line or intersects at least one second phantom line connecting two detectors except for the detectors forming the first phantom line.

### Advantageous Effects of Invention

According to the present disclosure, gas detection performance can be enhanced with a configuration that does not need an air current source.

### BRIEF DESCRIPTION OF DRAWINGS

FIG. 1 is a diagram illustrating a primary configuration of a gas sensor according to Embodiment 1 of the present disclosure.

FIG. 2 is a diagram illustrating one example of a configuration of a gas sensor element included in the gas sensor.

FIGS. 3A and 3B are diagrams illustrating a specific example of an arrangement of the gas sensor elements.

FIGS. 4A to 4E are perspective views of a housing that houses a substrate on which the gas sensor elements are arranged when seen from above.

FIGS. 5A to 5D are perspective views of each unit when the housing is seen from the same direction as that in FIG. 3B.

FIG. 6 is a diagram illustrating a primary configuration of a gas sensor according to Embodiment 2 of the present disclosure.

FIG. 7A is a diagram illustrating an arrangement of gas sensor elements. FIG. 7B is a graph showing intensity and detection time of a detection signal when an air current including gas as a detection target flows in from a direction indicated by an arrow in FIG. 7A. FIG. 7C is a graph when the detection intensity and the detection time shown in FIG. 79 are averaged.

FIG. 8A is a diagram illustrating a different flow of an air current in the same arrangement of the gas sensor elements as that in FIG. 7A. FIG. 8B is a graph showing intensity and detection time of a detection signal when an air current flows in from a direction indicated by an arrow in FIG. 8A. FIG. 8C shows a graph when the detection intensity and the detection time shown in FIG. 8B are averaged.

FIGS. 9A and 9B are diagrams illustrating a specific example of an arrangement of gas sensor elements included in a gas sensor according to Embodiment 3 of the present disclosure.

FIGS. 10A to 10C are perspective views of a housing that houses a substrate on which the gas sensor elements are arranged when seen from above.

FIGS. 11A to 11C are perspective views of each unit when the housing is seen from the same direction as that in FIG. 9B.

FIG. 12A is a diagram illustrating an arrangement of the gas sensor elements. FIG. 12B is a graph showing intensity and detection time of a detection signal when an air current including gas as a detection target flows in from a direction indicated by an arrow in FIG. 12A. FIG. 12C is a graph When the detection intensity and the detection time shown in FIG. 12B are averaged,

FIGS. 13A and 13B are diagrams illustrating a specific example of an arrangement of the gas sensor elements according to Embodiment 4 of the present disclosure.

FIGS. 14A and 14B are perspective views of a housing that houses a substrate on which the gas sensor elements are arranged when seen from above.

FIG. 15A is a diagram illustrating an arrangement of the gas sensor elements. FIG. 15B is a graph showing intensity and detection time of a detection signal when an air current including gas as a detection target flows in from a direction indicated by an arrow in FIG. 15A. FIG. 15C is a graph When the detection intensity and the detection time shown in FIG. 15B are averaged,

FIG. 16A. is a diagram illustrating an arrangement of the gas sensor elements. FIG. 169 is a graph showing intensity and detection time of a detection signal when an air current including gas as a detection target flows in from a direction indicated by an arrow in FIG. 16A. FIG. 16C is a graph when the detection intensity and the detection time shown in FIG. 16B are averaged.

FIGS. 17A to 17C are diagrams illustrating another example of an arrangement of gas sensor elements in a gas sensor according to Embodiment 5 of the present disclosure.

FIG. 18 is a diagram illustrating an arrangement example of gas sensor elements in a gas sensor in the related art.

### DESCRIPTION OF EMBODIMENTS

The present disclosure relates to a gas sensor serving as a functional unit for detecting gas included in an air current and achieves enhancement of gas detection performance by devising an arrangement position of a gas sensor element (detector) included in the gas sensor. In each embodiment, a configuration of the gas sensor according to the present disclosure, a method for arranging the gas sensor element, and processing using a detection result of the gas sensor element will be described below in detail.

### Embodiment 1

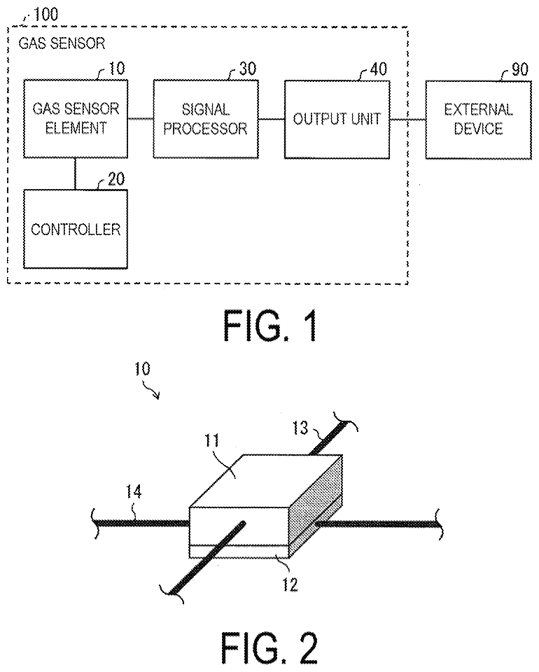

First, a primary configuration of a gas sensor 100 according to the present embodiment will be described with reference to FIG. 1.

### Primary Configuration of Gas Sensor

FIG. 1 is a diagram illustrating a primary configuration of the gas sensor 100 according to the present embodiment. The gas sensor 100 includes three or more gas sensor elements 10, a controller 20, a signal processor 30, and an output unit 40. Further, the gas sensor 100 is connected to an external device 90 via the output unit 40.

Note that it is assumed in the following description that the gas sensor 100 includes each one of the controller 20, the signal processor 30, and the output unit 40 for the plurality of gas sensor elements 10. In other words, the controller 20 collectively controls the plurality of gas sensor elements 10, the signal processor 30 pre-processes each electrical signal output from the plurality of gas sensor elements 10, and the output unit 40 outputs the electrical signals. Alternatively, the gas sensor 100 may include the controller 20, the signal processor 30, and the output unit 40 for each of the gas sensor elements 10. Further, the controller 20, the signal processor 30, and the output unit 40 may be provided separately from the gas sensor elements 10 as illustrated or may be provided integrally with the gas sensor elements 10.

It is also assumed in the following description that the gas sensor 100 collectively controls an action and an output concerned with gas detection of the gas sensor elements 10 having different gas response characteristics by the controller 20, the signal processor 30, and the output unit 40. However, the gas sensor 100 may separately include a controller 20, a signal processor 30, and an output unit 40 according to a type of gas response characteristics.

### Gas Sensor Element

The gas sensor element 10 is an element that reacts to prescribed gas. A type (component) of gas detected by the gas sensor element 10 is not particularly limited, but may be, for example, oxidation-reduction gas, flammable gas, volatile organic compounds (VOC) gas, or the like. More specifically, the gas sensor element 10 detects gas such as nitrogen oxide, sulfur oxide, carbon monoxide, carbon dioxide, hydrogen, methane, ethane, ethylene, propane, butane, methanol, ethanol, IPA, ammonia, formaldehyde, acetaldehyde, acetone, chloroform, isobutane, and hydrocarbon, for example.

Herein, a configuration of the gas sensor element 10 will be described with reference to FIG. 2. FIG. 2 is a diagram illustrating one example of the configuration of the gas sensor element 10 according to the present embodiment. Note that a configuration of a semiconductor gas sensor element 10 using an oxide semiconductor will be described as one example in FIG. 2. However, the configuration of the gas sensor element 10 is not limited to the configuration in FIG. 2, and configurations of various known gas sensor elements are applicable. For example, a combustion sensor element, an electrochemical sensor element, a stress detecting sensor element, an electrical capacitance detecting sensor element, and a field-effect transistor (FET) sensor element can be used as the gas sensor element 10.

The gas sensor element 10 includes a detection body 11 formed of oxide semiconductor mainly containing tin oxide and zinc oxide, a heater 12 that heats the detection body 11, a heater line 14 for supplying power to the heater, and an electric conductor 13 that energizes the detection body 11.

The detection body 11 is heated by heat emitted from the heater 12 and also energized by the electric conductor 13. When gas as a detection target of the gas sensor element 10 reaches the detection body 11 in this state, an oxidation-reduction reaction occurs on an oxide semiconductor surface of the detection body 11 and then an electrical resistance value of the detection body 11 changes, Therefore, when gas reaches the detection body 11 (the detection body 11 touches gas) while the detection body 11 is energized at a fixed voltage, a value of a current flowing through the electric conductor 13 changes. The gas sensor element 10 outputs this current value to the external device 90 via the signal processor 30 and the output unit 40 connected to the electric conductor 13. The external device 90 that receives an input of the current value can detect gas from the current value and specify a concentration of gas and a speed of an air current including the gas.

The gas sensor element 10 can change a gas response characteristic (namely, a type of detectable gas and a degree of change in the resistance value during detection) by changing a material and a composition of oxide semiconductor constituting the detection body 11 and adding an additive element or the like. A filter capable of preventing passage of some gas or reducing a concentration of some gas without changing a material and a composition of the detection body 11 may be provided so as to cover the detection body 11. By providing the above-described filter, the gas sensor element 10 having a gas response characteristic that is substantially different from that in a case without the filter can be obtained. The gas sensor element 10 having a different gas response characteristic can also be obtained by changing a gas detection method and a heater temperature.

The controller 20 performs various types of control to bring the gas sensor element 10 into a state where the gas sensor element 10 can react to gas and various types of control to detect the reaction as a signal. In the present embodiment, the controller 20 controls energization of the electric conductor 13. Also in the present embodiment, the controller 20 controls a degree of heat from the heater 12 by controlling energization of the heater line 14. Note that the controller 20 may perform control according to a configuration (gas detection method) of the gas sensor element 10. For example, in a case where the gas sensor element 10 can detect gas without a need for energization such as a case where the gas sensor element 10 is an electrochemical gas sensor element, the controller 20 need not perform energization control.

The signal processor 30 pre-processes an electrical signal obtained from the gas sensor element 10 in preparation for an output from the output unit 40. The signal processor 30 performs, for example, filtering for reducing noise, baseline correction, analog-digital conversion, and the like. The signal processor 30 transmits the pre-processed electrical signal to the output unit 40.

The output unit 40 outputs the electrical signal pre-processed by the signal processor 30 to the external device 90. Note that the output unit 40 may output the above-described electrical signal to the external device 90 in any of wired and wireless fashions. The external device 90 uses an electrical signal input from the output unit 40, namely, a result of gas detection of the gas sensor 100.

### Arrangement Example of Gas Sensor Element

Next, a method for arranging the gas sensor elements 10 in the gas sensor 100 will be described. The gas sensor 100 includes three or more gas sensor elements 10 as mentioned above. Two or more gas sensor elements 10 of the three or more gas sensor elements 10 have substantially the same gas response characteristic.

Furthermore, the gas sensor elements 10 are arranged such that a phantom line (first phantom line) connecting two gas sensor elements 10 of the gas sensor elements 10 having substantially the same gas response characteristic intersects another gas sensor element 10 except for the gas sensor elements 10 forming the first phantom line or intersects at least one phantom line (second phantom line) formed by two gas sensor elements 10 except for the gas sensor elements 10 forming the first phantom line. Note that when the gas sensor element 10 has a size large (an area of a detection surface) to some extent, a phantom line can be formed by, for example, connecting the center or barycenter of a detection surface of a certain gas sensor element 10 to the center or barycenter of a detection surface of another gas sensor element 10.

Note that “substantially the same gas response characteristic” means that a difference in gas response characteristic among the gas sensor elements 10 is within a range of error of detection sensitivity of the element. For example, when a deviation in detection value between the gas sensor elements 10 is less than or equal to 10%, the gas sensor elements 10 may be assumed to have substantially the same gas response characteristic. More specifically, instead of setting a threshold of 10% as a deviation in detection value between the gas sensor elements 10 to determine whether a gas response characteristic is the same or different, when a first sensor group of the gas sensor elements 10 provided in the gas sensor has a certain gas response characteristic and a second sensor group of the gas sensor elements 10 can detect a response different from. that of the first sensor group, the gas sensor element 10 belonging to the first sensor group and the gas sensor element 10 belonging to the second sensor group may be assumed to have different gas response characteristics. Note that in the following description, when the gas sensor elements 10 have substantially the same gas response characteristic, it will be simply described that the gas sensor elements 10 “have the same gas response characteristic” including a case where the gas sensor elements 10 that can be considered as one sensor group have a gas response characteristic.

According to the above-described configuration, a gas type can he determined by using a signal detected by a detector having the same gas response characteristic present on the first phantom line and a signal detected by a detector having another gas response characteristic. Herein, an average value of detection values of two detectors having the same gas response characteristic roughly coincides with a detection value when one gas sensor element having the same gas response characteristic is arranged at a midpoint between those detectors. Therefore, although detectors cannot be arranged in the same position or a position where a part thereof overlap each other in reality, a detection value detected when the detectors are simulatively arranged in the same position can be calculated. Therefore, gas detection can be performed more accurately, and gas detection performance can thus be enhanced without an air current source,

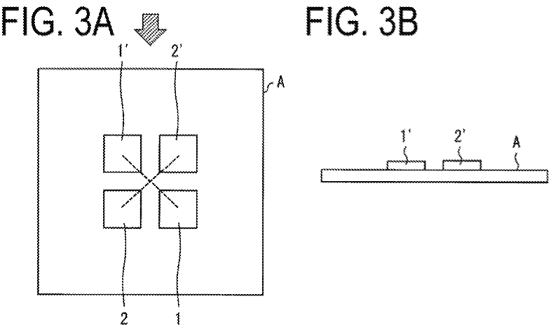

A more specific example of an arrangement of the gas sensor elements 10 according to the present embodiment will be described below with reference to FIGS. 3A and 3B. FIGS. 3A and 3B are diagrams illustrating a specific example of the arrangement of the gas sensor elements 10 in the gas sensor 100 according to the present embodiment. 1, 1′, 2, and 2′ in FIGS. 3A and 3B each indicate one gas sensor element 10. The gas sensor elements 1 and 1′ are the gas sensor elements 10 having the same gas response characteristic. Further, the gas sensor elements 2 and 2′ have the same gas response characteristic that is different from that of the gas sensor elements 1 and 1′.

FIG. 3A illustrates an arrangement of the gas sensor elements 1, 1′, 2, and 2′ arranged on a substrate A when seen from a direction that is vertical to a gas detection surface (namely, a surface parallel to the substrate A) and faces the gas detection surface. On the other hand, FIG. 3B illustrates an arrangement of the gas sensor elements when the gas sensor elements (and the substrate A) are seen from a direction of an arrow illustrated in FIG. 3A and a direction parallel to the gas detection surface of each of the gas sensor elements (and the substrate A). Note that in the following description, a direction that is vertical to the gas detection surface as in FIG. 3A and goes upward from the heater 12 of the gas sensor element 10 to the gas detection surface is referred to as an “upward direction”, and each direction parallel to the gas detection surface is referred to as a “lateral direction”.

It is assumed that all the gas sensor elements 10 are arranged on a single substrate (substrate A) in the arrangement example of the gas sensor elements 10 illustrated in FIGS. 3A and 3B and subsequent drawings. However, the gas sensor 100 may achieve the arrangement of the gas sensor elements 10 as illustrated in FIGS. 3A and 3B by disposing the plurality of gas sensor elements 10 on different substrates and combining the substrates. The same applies to the subsequent drawings.

As illustrated in FIG. 3A, the gas sensor 100 according to the present embodiment includes a total of two groups of two gas sensor elements 10 having the same gas response characteristic. As indicated by broken lines in FIG. 3A, the gas sensor elements are arranged such that a phantom line connecting the gas sensor elements 1 and 1′ having the same gas response characteristic intersects a phantom line connecting another group of the gas sensor elements 2 and 2′ having the same gas response characteristic. Note that the phantom line is an auxiliary line for description and not actually displayed. The same applies to the subsequent drawings.

The gas sensor elements (1, 1′, 2, and 2′) are arranged in the arrangement as illustrated in FIGS. 3A and 3B, so that a gas type can be determined by using an electrical signal detected by the gas sensor elements (for example, 1 and 1′) having the same gas response characteristic and an electrical signal detected by the other gas sensor elements (for example, 2 and 2′) having the same gas response characteristic.

Herein, an average value of detection values of two gas sensor elements having the same gas response characteristic roughly coincides with a detection value when one gas sensor element having the same gas response characteristic is arranged at a midpoint between those gas sensor elements, Therefore, an average value of the detection values of the gas sensor elements 1 and 1′ coincides with a detection value of a gas sensor element that has the same gas response characteristic as that of the gas sensor elements 1 and 1′ and is arranged at a midpoint of the phantom line of the gas sensor elements 1 and 1′ indicated by the broken line in FIG. 3A. The same also applies to the gas sensor elements 2 and 2′.

As illustrated in 3A, the phantom line of the gas sensor elements 1 and 1′ intersects the phantom line of the gas sensor elements 2 and 2′. Therefore, an average value of the detection values of the gas sensor elements 1 and 1′ and an average value of the detection values of the gas sensor elements 2 and 2′ are calculated simulatively from a value detected when two types of gas sensor elements having different gas response characteristics that cannot be arranged in the same position in reality are arranged in the same position.

In this way, false detection of gas due to displacement of gas sensor elements having different gas response characteristics caused by, for example, arranging them next to each other can be reduced. A deviation in detection value due to the displacement can be compensated by taking an average of signals detected by a plurality of gas sensor elements without a complicated computation process.

Note that in addition to the arrangement illustrated in FIGS. 3A and 3B, the same effect can also be obtained when the gas sensor elements 10 are arranged such that, as mentioned above, the first phantom line intersects a gas sensor element 10 having a gas response characteristic different from that of the gas sensor elements 10 forming the first phantom line. In other words, an average value of the detection values of the two gas sensor elements 10 forming the first phantom line roughly coincides with a detection value when the gas sensor element 10 is arranged at a midpoint of the first phantom line. Therefore, the gas sensor element 10 having a different gas response characteristic is arranged in the position intersecting the first phantom line, and thus a value detected when two types of gas sensor elements 10 having different gas response characteristics that cannot be arranged in the same position or in the position to overlap each other in reality, for example, are arranged in the same position can be simulatively calculated. Therefore, gas detection performance can be further enhanced.

A direction from which an air current including gas flows in and a speed of the air current can be determined from at least one of a difference in detection value (detection intensity) and a difference in detection time between two gas sensor elements having the same gas response characteristic. Specifically, an average value of the detection intensity between the gas sensor elements 1 and 1′ coincides with detection intensity of a gas sensor element that has the same gas response characteristic as that of the gas sensor elements 1 and 1′ and is arranged at a midpoint of the phantom line of the gas sensor elements 1 and 1′ indicated by the broken line in FIG. 3A. The same also applies to the gas sensor elements 2 and 2′.

Note that the number of gas sensor elements 10 having the same gas response characteristic may be any number that is two or more in the present embodiment. For example, the gas sensor 100 may include a gas sensor element 1″ having the same gas response characteristic as that of the gas sensor elements 1 and 1′. In this case, gas sensor elements may be arranged such that a phantom line connecting three (or more) gas sensor elements having the same gas response characteristic intersects a gas sensor element having a different gas response characteristic or intersects a phantom line different from the above-described phantom line. More specifically, when gas sensor elements are classified into groups according to gas response characteristics, the numbers of gas sensor elements in groups need not be identical to each other. Specifically, the gas sensor 100 may include the above-mentioned gas sensor elements 1, 1′, and 1″ and the gas sensor elements 2 and 2′.

Positional Relationship between Gas Sensor Element and Opening

The gas sensor 100 according to the present embodiment may be achieved by a gas detection device that causes an air current including gas as a detection target to flow in and out and is housed in a housing having at least one opening.

Note that the “housing” may be a housing that houses only the gas sensor 100 or may be a housing of a device including the gas sensor 100 (a single product including the gas sensor 100, the external device 90, and the like). Note that it is assumed in the following description that the housing is a housing that houses only the gas sensor 100.

The opening need not be clearly formed as an inflow port and an outflow port. For example, a place formed by a gap through which a molecule of gas as a detection target can substantially flow in and out or formed of a member having gas permeability (a member having a mesh structure, a filter member allowing passage of an air current, or a member having a punched hole) in a surface of the housing may be provided and serve as an opening.

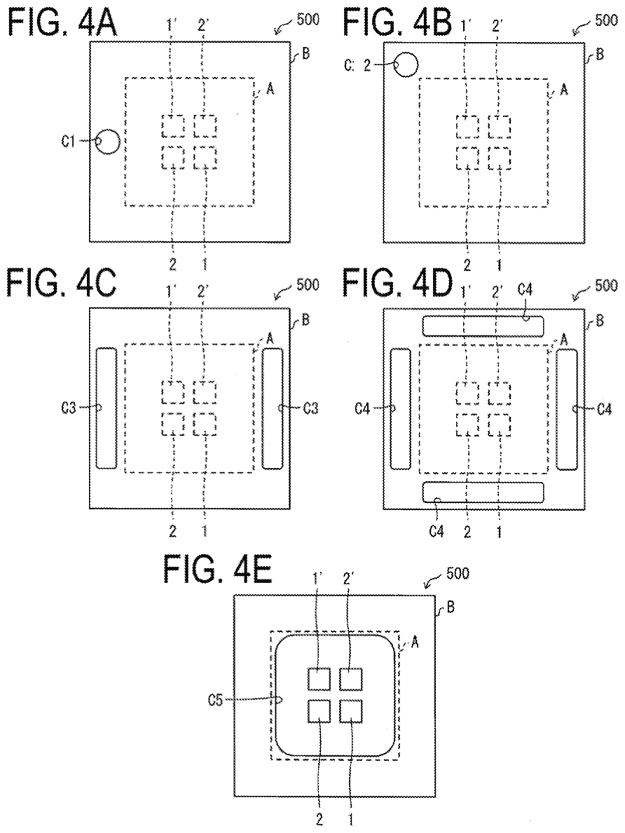

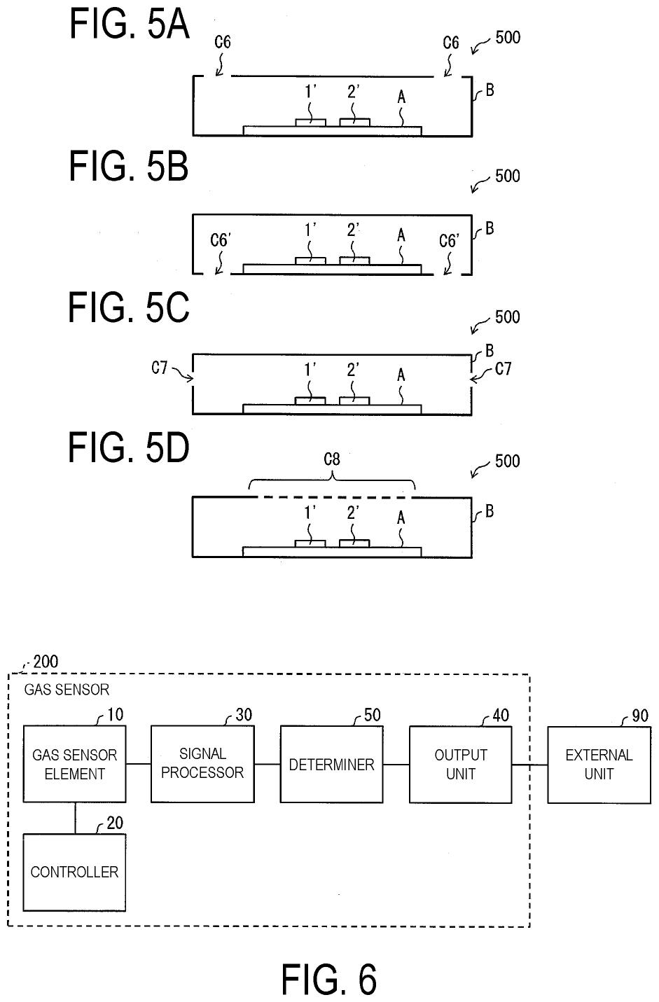

A positional relationship between the gas sensor 100, and the housing and opening will be described below with reference to FIGS. 4A to 5D. FIGS. 4A to 4E are perspective views of a gas detection device 500 including a housing B that houses the substrate A on which the gas sensor elements 1, 1′, 2 and 2′ are arranged when seen from above. FIGS. 5A to 5D are perspective views of each unit when the housing B is seen from the same direction as that in FIG. 3B (lateral direction from the direction of the arrow illustrated in FIG. 3A). Broken lines in the drawings indicate positions of members that are actually hidden by the housing B and unseen.

As illustrated in FIGS. 4A to 4E and FIGS. 5A to 5D, the housing 13 has at least one opening. A position of the opening in the housing B is not particularly limited, but as illustrated in FIGS. 4A and 4B, for example, one circular opening C1 or C2 may be provided in a region outside the substrate A when the housing B is seen from above. Further, as illustrated in FIGS. 4C, 4D, and 5A, for example, a pair of openings C3 may be provided in left and right regions or top and bottom regions outside the substrate A when the housing B is seen from above, or openings C4 may be provided in all top, bottom, left, and right regions outside the substrate A.

The detection surfaces of the gas sensor elements 1, 1′, 2, and 2′ need not be covered by the housing. For example, as illustrated in FIG. 4E, the detection surfaces of the gas sensor elements 1, 1′, 2, and 2′ may be exposed to the outside by providing an opening C5 in a surface of the housing B located above the substrate A (upper surface of the housing B), As illustrated in FIG. 5D, an opening C8 formed of a member having a mesh structure, a filter member allowing passage of an air current, or a member having a punched hole may be provided in a surface located above the substrate A (upper surface of the housing B).

As illustrated in FIG. 5C, openings C7 may be provided in surfaces of the housing B located in an orthogonal direction to the substrate A (side surfaces of the housing B). Furthermore, the arrangement of the openings C1 to C0, C6′, C7, and C8 illustrated in FIGS. 4A to 4E and FIGS. 5A to 5D may be arbitrarily combined.

An opening in the housing B is preferably formed in a region outside all of gas sensor elements and substantially in a parallel direction (lateral direction) with respect to gas detection surfaces (and the substrate A). In this way, an air current flows from the lateral direction with respect to a gas detection surface, so that a direction and a speed of the air current can be more accurately detected by using a detection result of each gas sensor element in comparison with a case where an air current flows in from above.

An opening is formed in a region outside all of the gas sensor elements 10 and substantially in a parallel direction (lateral direction) with respect to the gas detection surfaces (and the substrate A) in such a manner, so that an air current can flow in and out at a stable speed in an inflow direction with respect to the gas sensor elements 10 without an air current source. Therefore, gas detection performance of each of the gas sensor elements 10 can be enhanced.

When a direction and a speed of an air current flowing in and out from an opening is detected by using detection results of the gas sensor elements 1, 1′, 2, and 2′, the housing 13 preferably has a width of space in a parallel direction (lateral direction) to the substrate A greater than a width (thickness) of space in a vertical direction (up-and-down direction) to the substrate A. The details will be described later.

In this way, an opening provided in the housing B allows an air current to flow at a stable speed in an inflow direction with respect to the gas sensor 100 without an air current source, Therefore, gas detection performance in the gas sensor 100 can be enhanced.

### Embodiment 2

A gas sensor according to the present disclosure may include a first calculator that calculates an average value of at least one of intensity (detection value) and detection time of gas detection of at least one pair of two gas sensor elements 10 that are connected to each other with a phantom line and have the same gas response characteristic,

The gas sensor according to the present disclosure may further include a second calculator that calculates a difference in at least one of intensity and detection time of gas detection of at least one pair of two gas sensor elements 10 that are connected to each other with a phantom line and have the same gas response characteristic.

An embodiment of the present disclosure will he described below with reference to FIGS. 6 to 8C. Note that, for convenience of explanation, components illustrated in respective embodiments are designated by the same reference numerals as those having the same function, and the descriptions of these components will be omitted. The same also applies to the following embodiment.

FIG. 6 is a diagram illustrating a primary configuration of a gas sensor 200 according to the present embodiment. The gas sensor 200 is different from the gas sensor 200 according to Embodiment 1 in that the gas sensor 200 includes a determiner 50 (the first calculator and the second calculator).

The determiner 50 calculates an average value of detection value (detection intensity) and detection time and a difference in the detection intensity and the detection time of two gas sensor elements 10 having the same gas response characteristic. The determiner 50 specifies a detection value of each of the gas sensor elements 10 from an electrical signal received from a signal processor 30, and specifies detection time of each of the gas sensor elements 10 from timing for receiving the electrical signal. Then, the above-described average value and difference of a combination of the gas sensor elements 10 having the same gas response characteristic previously stored in the determiner 50 are calculated.

Furthermore, the determiner 50 determines at least any of a type of gas and a direction and a speed of an air current from the above-described average value and difference, and outputs a determination result to an external device 90 via an output unit 40. Note that information output from the determiner 50 need not necessarily include information directly related to a type of gas and a direction and a speed of an air current, and may be secondary information that can be determined from these pieces of information.

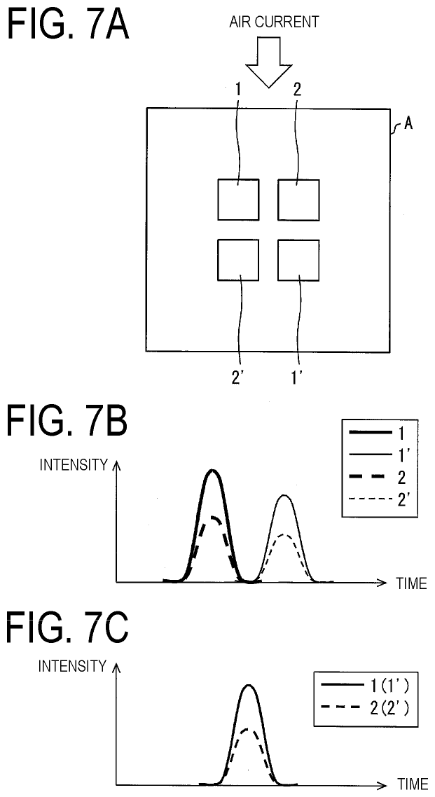

FIG. 7A illustrates an arrangement of gas sensor elements 1, 1′, 2, and 2′ arranged on a substrate A when seen from above gas detection surfaces. Note that a gas response characteristic of the gas sensor elements 1, 1′, 2, and 2′ is the same as that described in Embodiment 1. FIG. 7B is a graph showing intensity of a detection signal in the gas sensor elements 1, 1′, 2, and 2′ in a y-axis and detection time thereof in an x-axis when an air current including gas as a detection target flows in from a lateral direction to the gas sensor elements 1, 2, and 2′ and a direction indicated by an arrow in FIG. 7A. It is described in the following description on the assumption that the gas sensor element 1 has a greater response to gas as a detection target (greater detection intensity) than that of the gas sensor element 2.

When an air current flows in from the direction of the arrow illustrated in FIG. 7A, first, the gas sensor elements 1 and 2 located on an upstream side in the air current direction react to gas approximately at the same time as shown in FIG. 79. Subsequently, after the reaction of the gas sensor elements 1 and 2, the gas sensor elements 1′ and 2′ react approximately at the same time. At this time, detection intensity detected by the gas sensor elements 1′ and 2′ is lower than detection intensity detected by the gas sensor elements 1 and 2 due to diffusion of the air current. Note that “react approximately at the same time” herein indicates that gas sensor elements react substantially at the same time, inclusive of a detection speed and a detection error of each gas sensor element.

The determiner 50 specifies detection results of the gas sensor elements 1, 1′, 2, and 2′ as shown in FIG. 7B from an electrical signal received from the signal processor 30. Then, the determiner 50 averages detection intensity and determination time of each pair of the gas sensor elements 1 and 1′ and the gas sensor elements 2 and 2′ having the same gas response characteristic.

FIG. 7C shows a graph when the detection intensity and the detection time of each of the gas sensor elements shown in FIG. 7B are averaged. An average value thereof simulatively indicates detection intensity and detection time of a gas sensor element that has the same gas response characteristic as that of the gas sensor elements 1 and 1′ and is arranged at a midpoint of a phantom line of the gas sensor elements 1 and 1′ (the same also applies to the gas sensor elements 2 and 2′). Therefore, the gas sensor 200 can specify, from an average value calculated by the determiner 50, behavior detected when two types of gas sensor elements (1 and 1′ and 2 and 2′) having different gas response characteristics that cannot be arranged in the same position in reality are arranged in the same position. In this way, false determination can be prevented by correcting a deviation in detection intensity and detection time caused by displacement of an arrangement position of each gas sensor element, and gas detection performance can be enhanced.

When the determiner 50 receives detection results of the gas sensor elements 1, 1′, 2, and 2′ as shown in FIG. 79 from the signal processor 30, the determiner 50 may calculate at least one of a difference in detection intensity and a difference in detection time between the gas sensor elements 1 and 1′ and the gas sensor elements 2 and 2′ having the same gas response characteristic. A direction and a speed of an air current including gas can be determined from at least one of a difference in the detection intensity and a difference in the detection time. Therefore, gas detection can be performed more accurately. A method for specifying a direction and a speed of an air current will be described below in detail.

When an air current flows from the direction of the arrow illustrated in FIG. 7A, first, the gas sensor elements 1 and 2 located on the upstream side in the air current direction react to gas approximately at the same time, and the gas sensor elements 1′ and 2′ then react approximately at the same time, as mentioned above. At this time, a difference in detection time between the gas sensor elements 1 and 1′ increases as a speed of the air current including gas is slower. Since which gas sensor element of the gas sensor elements 1 and 1′ reacts first can be determined by taking the difference in the detection time, which direction of the gas sensor element the air current flows from can be determined. The same also applies to the gas sensor elements 2 and 2′.

On the other hand, a difference in detection intensity between the gas sensor elements 1 and 1′ increases as a speed of the air current is slower. This is caused by diffusion of gas included in the air current. Since which gas sensor element of the gas sensor elements 1 and 1′ has greater detection intensity can be determined from the difference in the detection intensity between the gas sensor elements 1 and 1′, which direction of the gas sensor element the air current flows from can be determined. The same also applies to the gas sensor elements 2 and 2′.

In this way, the determiner 50 can determine a direction and a speed of an air current including gas by calculating at least one of a difference in detection intensity and a difference in detection time of various gas sensor elements arranged in the gas sensor 200. Furthermore, the determiner 50 can determine a direction and a speed of an air current more accurately by using a difference in detection intensity and a difference in detection time of more gas sensor elements arranged in the gas sensor 200 to determine a direction and a speed of the air current.

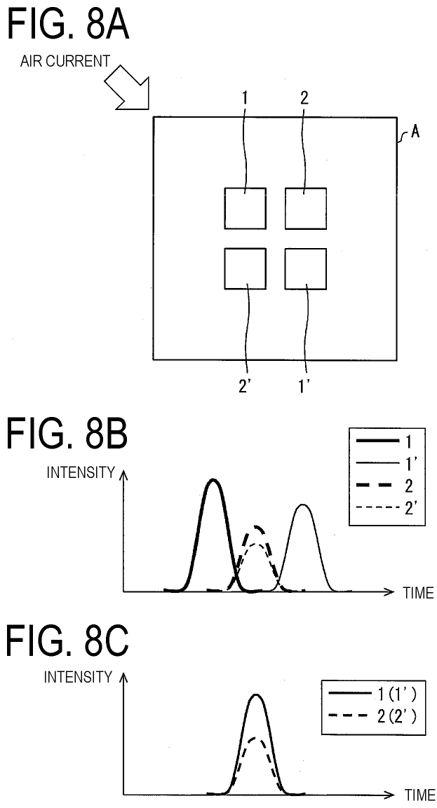

FIG. 8A is a diagram illustrating a flow of an air current from a lateral direction and a direction toward a corner of the substrate A (a direction of an arrow referred to as a diagonal direction) in the same arrangement of the gas sensor elements as that in FIG. 7A. FIG. 8B is a graph showing intensity of a detection signal in the gas sensor elements 1, 1′, 2, and 2′ in a y-axis and detection time thereof in an x-axis when an air current flows in from a direction indicated by an arrow in FIG. 8A. FIG. 8C shows a graph when the detection intensity and the detection time of each of the gas sensor elements shown in FIG. 8B are averaged.

In this way, when the air current flows in from the diagonal direction, the gas sensor element 1 first reacts to gas, and the gas sensor elements 2 and 2′ then react to the gas. At the end, the gas sensor element 1′ reacts to the gas. Therefore, the determiner 50 can also specify a direction of an air current from the above-mentioned diagonal direction accurately by using at least one of a difference in detection intensity and a difference in detection time of three or more gas sensor elements (for example, 1, 1′, and 2′) instead of two gas sensor elements (for example, 1 and 1′). Even when gas sensor elements having the same gas response characteristic are only two, for example, 1 and 1′, a speed of an air current in a direction orthogonal to a segment connecting the gas sensor elements 1 and 1′ can also be determined in consideration of a difference in detection intensity or a difference in detection time of a gas sensor element having a different gas response characteristic (for example, at least one of 2 and 2′, preferably both). Thus, the number of gas sensor elements 10 can be a minimum necessary number.

Note that the gas sensor element 10 may be a gas sensor element 10 having a high degree of selectivity (that reacts to only specific gas). However, information about detection intensity and detection time of one type of gas can be obtained from more gas sensor elements by setting the gas sensor elements 1 and 1′ and the gas sensor elements 2 and 2′ as the gas sensor elements 10 having different detection intensity according to a type of gas. Therefore, information for determining a direction and a speed of an air current increases, and the determiner 50 can thus determine a direction and a speed of the air current more accurately.

Note that when the gas sensor elements 10 are not equal in a. positional relationship, weighting correction in consideration of the amount of displacement may be performed. Specifically, it is assumed that only the gas sensor element 1 is arranged to be displaced from a state where the gas sensor elements 1, 1′, 2, and 2′ are arranged as illustrated in FIG. 7A or 8A, for example, to a direction away from the central position of each element (a direction diagonally to the upper left in FIG. 7A or 8A). When an air current flows from the direction illustrated in FIG. 7A or 8A in this state, timing of the gas sensor element 1 for detecting the air current is earlier than that in a case where the gas sensor elements are equally arranged as illustrated in FIG. 7A or 8A, and detection intensity of the gas sensor element 1 also increases, In this case, the amount of displacement from a position when the gas sensor elements are equally arranged is taken into consideration in a detection result of the gas sensor element 1, and weighting correction is then performed on detection timing (detection time) and intensity, Thus, the same output as that when the gas sensor elements are equally arranged can be obtained.

On the other hand, when an air current flows in a direction opposite to the direction of the air current illustrated in FIG. 7A or 8A, detection timing of the gas sensor element 1 arranged to be displaced is conversely late, and detection intensity thereof decreases. Thus, the above-described weighting correction is preferably performed by determining a weighting direction (code) based on a relation with a detection signal of the gas sensor element 1′ having the same gas response characteristic as that of the gas sensor element 1, that is, which gas sensor element reacts first or which gas sensor element has great response intensity.

Note that the gas sensor 200 may detect a direction and a speed of an air current more specifically by arranging the gas sensor elements 10 in matrix on the substrate, obtaining a detection value from each of the gas sensor elements 10, and allowing the determiner 50 to perform the above-mentioned processing. The gas sensor 200 may include a display as the external device 90 and allow the display to visually display, with one gas sensor element 10 acting as one pixel, information about a type of gas and a direction and a speed of an air current obtained by processing of the determiner 50.

### Embodiment 3

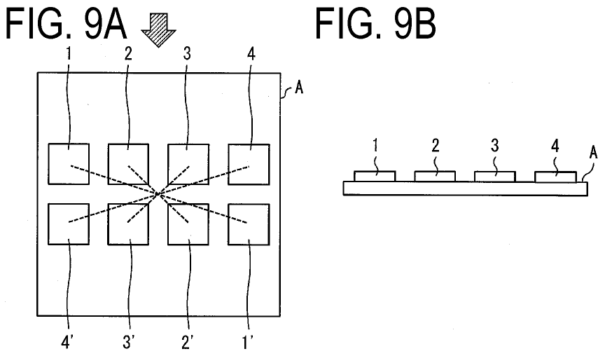

In the gas sensor 100 or 200 according to the present disclosure, the gas sensor elements 10 that are the gas sensor elements 10 forming the first phantom line or the second phantom line and each have a different gas response characteristic may be arranged in one line. An embodiment of the present disclosure will be described below with reference to FIGS. 9A to 12C.

FIGS. 9A and 9B are diagrams illustrating a specific example of an arrangement of the gas sensor elements 10 included in the gas sensor 100 or 200 according to the present embodiment. 1, 1′, 2, 2′, 3, 3′, 4, and 4′ in FIG. 9 and the subsequent drawings each indicate one gas sensor element 10. Note that it is assumed in the following description that the gas sensor elements 1, 2, 3, and 4 have the same gas response characteristic as that of the gas sensor elements 1′, 2′, 3′, and 4′, respectively. The gas sensor elements 1 (1′), 2 (2′), 3 (3′), and 4 (4′) have different gas response characteristics from each other.

FIG. 9A illustrates an arrangement of the gas sensor elements 1 to 4 and 1′ to 4′ arranged on a substrate A when seen from above gas. On the other hand, FIG. 9B illustrates an arrangement of the gas sensor elements when the gas sensor elements (and the substrate A) are seen from a lateral direction and a direction of an arrow illustrated in FIG. 9A. As illustrated in FIG. 9A, the gas sensor elements 1 to 4 according to the present embodiment are aligned straight and the gas sensor elements 1′ to 4′ having the same response are aligned in a reverse order so as to face the gas sensor elements 1 to 4. Note that the arrangement of the gas sensor elements is not limited to the reverse order.

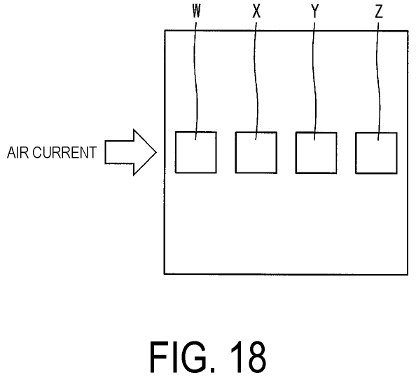

In this way, the gas sensor elements 1 to 4 are arranged in one line, and the gas sensor elements 1′ to 4′ having the same gas response characteristic as that of those respective gas sensor elements are arranged so as to face the gas sensor elements 1 to 4 and arranged such that any of two phantom lines (preferably, all phantom lines as illustrated in FIG. 9A) intersect each other. Thus, a detection value when a combination of the gas sensor elements having the phantom lines intersecting each other is arranged in the same position as a position in which a main body cannot be arranged can be calculated simulatively in comparison with a case where the gas sensor elements 1 to 4 are arranged individually (such as the case of the arrangement in FIG. 18). Therefore, false detection can be reduced, and gas detection performance can be enhanced. As described in the above-described embodiments, a direction and a speed of an air current including gas can be calculated by using a combination of gas sensor elements having phantom lines intersecting each other.

Note that as illustrated in FIG. 9A, the gas sensor 100 or 200 according to the present disclosure preferably includes three or more types of the gas sensor elements 10 having different gas response characteristics. A known multivariate analysis such as a principal component analysis, an independent component analysis, and a cluster analysis is applied by using three or more types of the gas sensor elements 10, so that a type of gas can be identified. Further, an increase in types of the gas sensor elements 10 increases the accuracy of identifying gas, and false detection can be reduced. Note that also in the present embodiment, the maximum number of types of the gas sensor elements 10 provided in the gas sensor 100 or 200 and the maximum number of the gas sensor elements are not particularly

As illustrated in FIG. 9, phantom lines of three or more types of gas sensor elements preferably intersect each other at one point. In this way, a detection value of each of the gas sensor elements when all the gas sensor elements are arranged at the one point can be calculated simulatively. Therefore, values of all the gas sensor elements under the same gas detection condition can be specified, so that gas detection can be performed more accurately. Further, a direction and a speed of an air current including gas can also he specified more accurately.

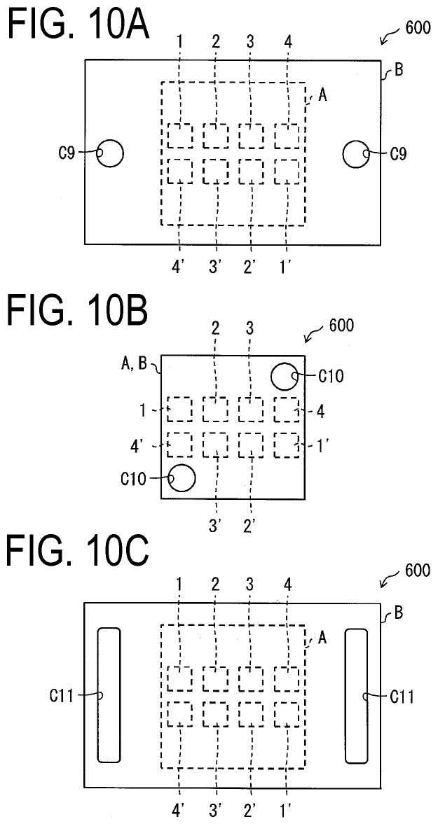

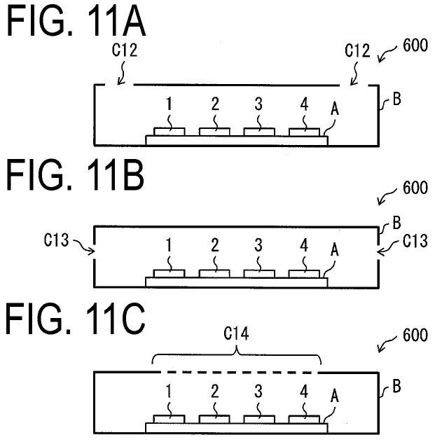

Note that the gas sensor (100 or 200) according to the present embodiment may also be housed in a housing having an opening formed in substantially a parallel direction with respect to a detection surface of the gas sensor element in a region outside the gas sensor element 10. FIGS. 10A to 10C are perspective views of a gas detection device 600 including a housing B that houses a substrate A on which the gas sensor elements 1 to 4 and 1′ to 4′ are arranged when seen from above. FIGS. 11A to 11C are perspective views of each unit when the housing B is seen from the same direction as that in FIG. 9B.

A position, a shape, and a size of an opening in the housing B are not particularly limited, but as illustrated in FIGS. 10A to 10C, for example, a pair of circular openings may be provided in a region outside the substrate A when the housing B is seen from above (openings C9 to C13). In the present embodiment, as illustrated in FIG. 11B, it is particularly preferable that openings are formed in a direction parallel to an arrangement direction of the gas sensor elements 1 to 4 (and 1′ to 4′) in terms of detecting a direction and a speed of an air current.

For example, as illustrated in FIG. 11A, openings C13 may be provided in surfaces of the housing B located in an orthogonal direction to the substrate A (side surfaces of the housing B). As illustrated in FIG. 11C, an opening C14 formed of a member having a mesh structure, a filter member allowing passage of an air current, or a member having a punched hole may be provided in a surface located above the substrate A (upper surface of the housing B). Furthermore, the arrangement of the openings C9 to C14 illustrated in FIGS. 10A to 10C and FIGS. 11A to 11C may be arbitrarily combined.

Note that it is assumed that a pair of two gas sensor elements having the same gas response characteristic is arranged in the arrangement example of FIG. 9, but the gas sensor 100 or 200 according to the present embodiment may include three or more gas sensor elements having the same gas response characteristic.

Note that the gas sensor according to the present embodiment may also be the gas sensor 200 including the determiner 50. Then, an average value of detection intensity and detection time and a difference in the detection intensity and the detection time of two gas sensor elements 10 having the same gas response characteristic may be calculated. 12A illustrates an arrangement of the gas sensor elements (1 to 4 and 1′ to 4′) of the gas sensor 200 arranged on the substrate A when seen from above gas detection surfaces.

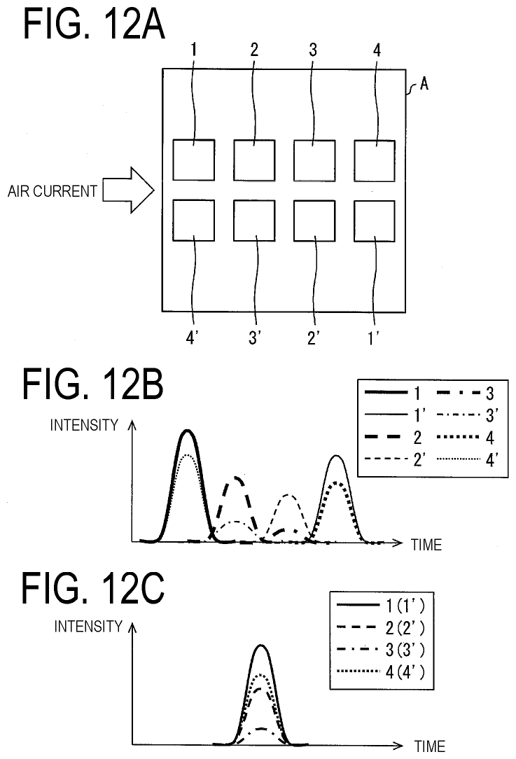

FIG. 12B is a graph showing intensity and detection time of a detection signal when an air current including gas as a detection target flows in from a lateral direction to the gas sensor elements and a direction indicated by an arrow in FIG. 12A. FIG. 12C is a graph when the detection intensity and the detection time shown in FIG. 12B are averaged. As shown in FIGS. 12A to 12C, magnitude of detection intensity of each of the gas sensor elements is 1 (1′)>4 (4′)>2 (2′)>3 (3′).

When an air current flows in from the direction of the arrow illustrated in FIG. 12A, first, the gas sensor elements 1 and 4′ located on an upstream side in the air current direction react to gas approximately at the same time as shown in FIG. 1213. Subsequently, after the reaction of the gas sensor elements 2 and 3′, the gas sensor elements 3 and 2′ and the gas sensor elements 4 and 1′ react in this order, At this time, detection intensity detected by the gas sensor elements on a downstream side is lower than detection intensity detected by the gas sensor elements on the upstream side due to diffusion of the air current.

Also in the present embodiment, as illustrated in FIG. 12B, the determiner 50 calculates an average value (FIG. 12C), a difference in detection intensity, and a difference in detection time from a value of gas detection intensity and detection time obtained from each of the gas sensor elements, so that gas detection (identification of a type of gas) may be performed, and a direction and a speed of an air current may be determined,

### Embodiment 4

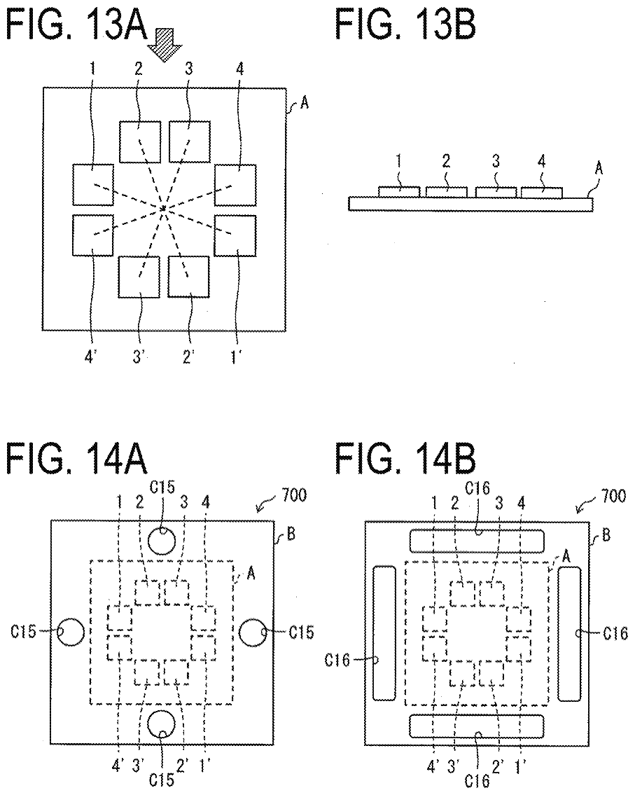

In a gas sensor 100 or 200 according to the present disclosure, gas sensor elements forming a first phantom line and gas sensor elements forming a second phantom line are preferably arranged on a circumference. An embodiment of the present disclosure will be described below with reference to FIGS. 13A to 16C.

FIGS. 13A and 13B are diagrams illustrating a specific example of an arrangement of gas sensor elements 10 included in the gas sensor 100 or 200 according to the present embodiment. FIG. 13A illustrates an arrangement of gas sensor elements 1 to 4 and 1′ to 4′ arranged on a substrate A when seen from above gas. On the other hand, FIG. 13B illustrates an arrangement of the gas sensor elements when the gas sensor elements (and the substrate A) are seen from a lateral direction and a direction of an arrow illustrated in FIG. 9A. As illustrated in FIG. 13A, the gas sensor elements 1 to 4 and 1′ to 4′ according to the present embodiment are arranged on the circumference so as to be displaced from each other not to be aligned straight. The gas sensor elements are arranged such that a phantom line connecting the gas sensor elements having the same gas response characteristic intersects another phantom line.

When the gas sensor elements are arranged on the same circumference in such a manner, an average value of detection intensity and detection time between the gas sensor elements (for example, 1 and 1′) having the same gas response characteristic can be calculated without complicated computation. Therefore, false detection can be reduced, and gas detection performance can be enhanced. As described in the above-described embodiments, a direction and a speed of an air current including gas can be calculated by using a combination of gas sensor elements having phantom lines intersecting each other.

Note that all the gas sensor elements according to the present embodiment are preferably arranged on the same circumference. As illustrated in FIG. 13A, all the phantom lines preferably intersect each other at one point. In this way, a detection value of each of the gas sensor elements when all the gas sensor elements are arranged at the one point can be calculated simulatively. Therefore, values of all the gas sensor elements under the same gas detection condition can be specified, so that gas detection can be performed more accurately. Further, a direction and a speed of an air current including gas can also be specified more accurately.

Since each of the gas sensor elements can be arranged in fine angular distribution at the time of an inflow of an air current, an air current direction can be more accurately identified by combining a response of a detector located in an orthogonal direction (at least a direction in which an axial direction is different) and responses of all elements to specify the air current direction, like a difference in detection intensity and a difference in detection time of each of the gas sensor elements.

Note that the gas sensor (100 or 200) according to the present embodiment may also be housed in a housing having an opening formed in substantially a parallel direction with respect to a detection surface of the gas sensor element in a region outside the gas sensor element 10, FIGS. 14A and 14B are perspective views of a gas detection device 700 including a housing B that houses a substrate A on which the gas sensor elements 1 to 4 and 1′ to 4′ are arranged when seen from above.

A position, a shape, and a size of an opening in the housing B are not particularly limited, and the position, the shape, and the size of the opening described in the above-mentioned embodiments may be adopted. However, the gas sensor 100 or 200 according to the present embodiment more preferably has a group of four openings C15 or C16 in left, right, top, and bottom regions outside the substrate A when the housing B is seen from above as illustrated in FIGS. 14A and 14B to clearly determine a direction of an air current, In this way, openings are provided in all sides of detection surfaces of the gas sensor elements, so that an air current can be introduced into the housing B without preventing an original direction of the air current from being changed.

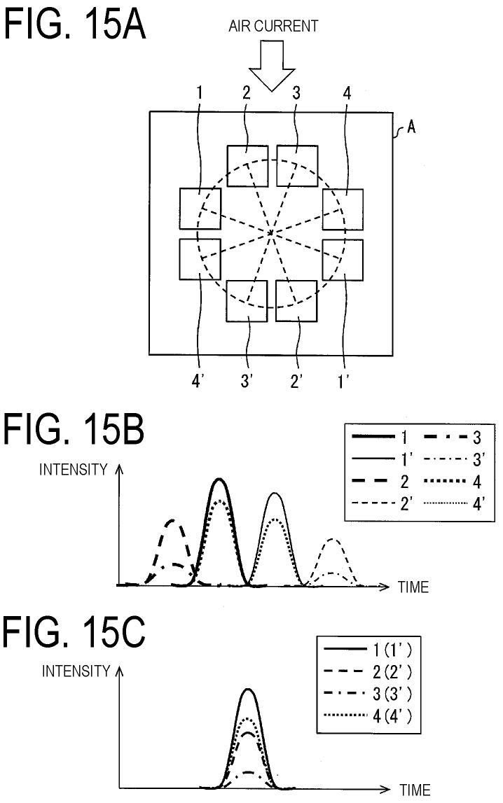

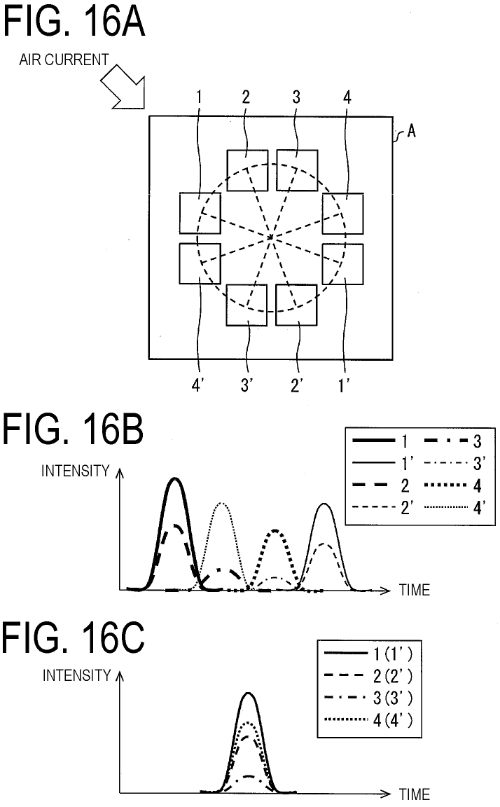

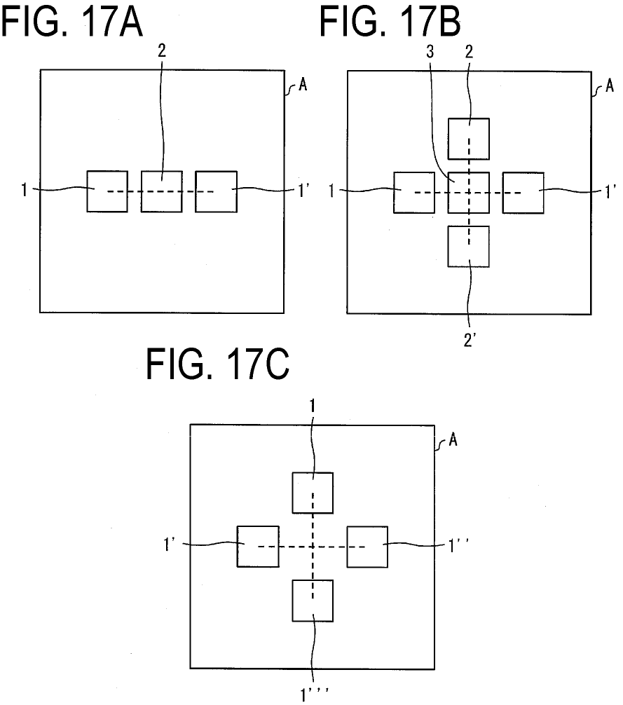

Note that the gas sensor according to the present embodiment may also be the gas sensor 200 including the determiner 50. Then, an average value of detection intensity and detection time and a difference in the detection intensity and the detection time of two gas sensor elements 10 having the same gas response characteristic may be calculated. FIGS. 15A and 16A illustrate an arrangement of the gas sensor elements (1 to 4 and 1′ to 4′) of the gas sensor 200 arranged on the substrate A when seen from above the gas detection surfaces.

FIGS. 15A and 16B are graphs showing intensity and detection time of a detection signal when an air current including gas as a detection target flows in from a lateral direction to the gas sensor elements and directions indicated by arrows in FIGS. 15A and 16A. FIGS. 15C and 16C are graphs when the detection intensity and the detection time shown in FIGS. 15B and 16B are averaged. As shown in FIGS. 15C and 16C similarly to FIG. 12C, magnitude of detection intensity of each of the gas sensor elements is 1 (1′)>4 (4′)>2 (2′)>3 (3′).

When an air current flows in from the direction of the arrow illustrated in FIG. 15A, first, the gas sensor elements 2 and 3 located on an upstream side in the air current direction react to gas approximately at the same time as shown in FIG. 15B. Subsequently, after the reaction of the gas sensor elements 1 and 4, the gas sensor elements 4′ and 1′ and the gas sensor elements 3′ and 2′ react in this order. At this time, detection intensity detected by the gas sensor elements 1′ to 4′ is lower than detection intensity detected by the gas sensor elements 1 to 4 due to diffusion of the air current.

On the other hand, when an air current flows in from the direction of the arrow illustrated in FIG. 16A, first, the gas sensor elements 1 and 2 located on an upstream side in the air current direction react to gas approximately at the same time as shown in FIG. 16B. Subsequently, after the reaction of the gas sensor elements 4′ and 3, the gas sensor elements 3′ and .4 and the gas sensor elements 2′ and 1′ react in this order. At this time, detection intensity detected by the gas sensor elements on a downstream side is lower than detection intensity detected by the gas sensor elements on the upstream side due to diffusion of the air current.

Also in the present embodiment, as illustrated in FIGS. 15B and 16B, the determiner 50 calculates an average value (FIGS. 15C and 16C), a difference in detection intensity, and a difference in detection time from a value of gas detection intensity and detection time obtained from each of the gas sensor elements, so that gas detection (identification of a type of gas) may be performed, and a direction and a speed of an air current may be determined.

### Embodiment 5

In the gas sensors 100 and 200 according to the present disclosure, the gas sensor elements 10 may be arranged as described below in addition to the arrangement presented in Embodiments 1 to 4. FIGS. 17A to 17C are diagrams each illustrating another example of an arrangement of the gas sensor elements 10 in the gas sensors 100 and 200.

For example, in the gas sensor 100 or 200 according to the present disclosure, as illustrated in FIG. 17A, the gas sensor elements may be arranged such that a phantom line (broken line) connecting gas sensor elements 1 and 1′ having the same gas response characteristic intersects a gas sensor element 2 having a gas response characteristic different from that of the gas sensor elements 1 and 1′. In a case of FIG. 17A, a result of averaging detection values of the gas sensor elements 1 and 1′ is a result detected by a gas sensor element having the same gas response characteristic as that of the gas sensor element 1 in the position of the gas sensor element 2. Therefore, false detection due to displacement between the gas sensor elements 1 and 1′ and the gas sensor element 2 can be reduced.

For example, in the gas sensor 100 or 200 according to the present disclosure, as illustrated in FIG. 17B, a gas sensor element 3 having a gas response characteristic different from that of the gas sensor elements 1, 1′, 2, and 2′ may be further arranged in a position in which a first phantom line connecting the gas sensor elements 1 and 1′ having the same gas response characteristic intersects a second phantom line connecting the gas sensor elements 2 and 2′ having the same gas response characteristic. In a case of FIG. 17B, results of averaging detection values of the gas sensor elements 1 and 1′ and the gas sensor elements 2 and 2′ are results detected by respective gas sensor elements having the same gas response characteristic as that of the gas sensor elements 1 and 2 in the position of the gas sensor element 3. Therefore, false detection due to displacement between the gas sensor elements 1 and 2 and the gas sensor element 3 can be reduced.

For example, in the gas sensor 100 or 200 according the present disclosure, as illustrated in FIG. 17C, gas sensor elements 1, 1′, 1″, and 1′″ having the same gas response characteristic may be arranged such that a phantom line connecting two of gas sensor elements intersects another phantom line as illustrated.

### Modification

Note that the gas sensors 100 and 200 according to the present disclosure may be provided together with an air current source. In other words, the present disclosure does not hamper an air current source being provided. When an air current source is provided, an air current may be generated by using a difference in temperature, a difference in atmospheric pressure, and introduction of carrier gas by, for example, a fan and heating. By providing an air current source and specifying a direction and a speed of an air current using the gas sensor 100 or 200, whether the air current source is normally operating may be determined. Note that the determination may be performed by the determiner 50 described in Embodiment 2 or the external device 90 described in Embodiment 1. Furthermore, at least one of a direction and intensity of an air current flowing in may be inevitably limited depending on a size and an arrangement of an opening instead of actively generating an air current by an air current source.

The present disclosure is not limited to each of the above-described embodiments. It is possible to make various modifications within the scope of the claims. An embodiment obtained by appropriately combining technical elements each disclosed in different embodiments falls also within the technical scope of the present disclosure. Furthermore, technical elements disclosed in the respective embodiments may be combined to provide a new technical feature.

### REFERENCE SIGNS LIST

10 Gas sensor element (detector)
20 Controller
30 Signal processor
40 Output unit
50 Determiner (first calculator, second calculator)
100, 200 Gas sensor
500, 600, 700 Gas detection device
A Substrate
B Housing
C1 to C16 Opening

1. A gas sensor comprising:
three or more detectors detecting gas included in an air current, wherein
two or more detectors of the detectors have substantially the same gas response characteristic, and
the detectors are arranged in such a way that a first phantom line connecting two detectors of the detectors having substantially the same gas response characteristic intersects another detector except for the detectors forming the first phantom line or intersects at least one second phantom line connecting two detectors except for the detectors forming the first phantom line.

2. The gas sensor according to claim 1, wherein
the second phantom line is a phantom line connecting two detectors having substantially the same gas response characteristic that is different from the gas response characteristic of the detectors forming the first phantom line.

3. The gas sensor according to claim 1, wherein
the other detector intersecting the first phantom line has a gas response characteristic different from the gas response characteristic of the detectors forming the first phantom line.

4. The gas sensor according to claim 1, wherein
a plurality of detectors that are detectors forming the first phantom line or the second phantom line and each have a different gas response characteristic are arranged in one line.

5. The gas sensor according to claim 1, wherein
the detectors forming the first phantom line and the detectors forming the second phantom line are arranged on a circumference.

6. The gas sensor according to claim 1, wherein
the detectors are arranged in such a way that the first phantom line intersects all the second phantom line at one point.

7. The gas sensor according to claim 1, wherein
the detectors include three or more types of detectors having different gas response characteristics.

8. The gas sensor according to claim 1, further comprising a first calculator calculating an average value of at least one of intensity and detection time of gas detection in at least the two detectors forming the first phantom line or the two detectors forming the second phantom line.

9. The gas sensor according to claim 1, further comprising a second calculator calculating at least one of a difference in intensity and a difference in detection time of gas detection in at least the two detectors forming the first phantom line or the two detectors forming the second phantom line.

10. A gas detection device comprising:
the gas sensor according to claim 1; and
a housing that houses the gas sensor.

11. The gas detection device according to claim 10, wherein
the housing has an opening formed in substantially a parallel direction with respect to a surface of each of the detectors for detecting gas in a region outside the detectors included in the gas sensor.
